(19) United States
(12) Patent Application Publication
(10) Pub. No.: US 2026/0301925 A1
(43) Pub. Date: Oct. 1, 2026

(54) METHODS FOR RECONSTRUCTION OF MRI IMAGE DATA

(71) Applicant: Siemens Healthineers AG, Forchheim (DE)

(72) Inventors: Karl-Philipp Kunze, London (GB); Radhouene Neji, London (GB)

(21) Appl. No.: 19/708,874

(22) Filed: Jun. 15, 2026

Related U.S. Application Data
(63) Continuation of application No. 17/722,270, filed on Apr. 15, 2022.

(30) Foreign Application Priority Data
Apr. 16, 2021 (GB) 2105423.4

(51) Int. Cl. G16H 30/40; A61B 5/055; G06T 7/00

(52) U.S. Cl. CPC G16H 30/40; A61B 5/055; G06T 7/0012; G06T 2207/10088; G06T 2207/30004

(57) ABSTRACT

A method for reconstructing a set of one or more MRI images from one or more segmented acquisitions of MRI raw data includes, for each respective shot capturing a part of the MRI raw data of the respective images, capturing and reconstructing a low-resolution navigation image of a region of interest, either from the part of the MRI raw data or from an additional MRI raw data acquired adjacent to the part of the MRI raw data in time. Each segmented acquisition consists of a number of shots.

This application is a continuation application of U.S. application Ser. No. 17/722,270, filed Apr. 15, 2022, which claims the benefit of UK Patent Application No. GB 2105423.4, filed on Apr. 16, 2021. These documents are hereby incorporated by reference in their entirety.

### SUMMARY

In one embodiment, a method for reconstructing a set of one or more magnetic resonance imaging (MRI) images from one or more segmented acquisitions of MRI raw data, each of the one or more segmented acquisitions including a plurality of shots, is provided. For each respective shot of the plurality of shots capturing a part of the MRI raw data of the respective MRI images, a low-resolution navigation image of a region of interest is captured and reconstructed, either from the part of the MRI raw data or from additional MRI raw data acquired adjacent to the part of the MRI raw data in time. Navigation images are clustered from the plurality of shots within each MRI image of the one or more MRI images according to a characteristic of the navigation images. A navigation image of the navigation images and a corresponding part of the MRI raw data are excluded from further processing when the navigation image is identified as an outlier in the clustering. Motion values of the object of interest are estimated from each remaining navigation image with respect to a reference navigation image within the respective cluster. The motion values are normalized for all data clusters across the one or more MRI images within the acquired set of one or more MRI images. A final reconstruction of the set of one or more MRI images is performed using the motion values.

### BRIEF DESCRIPTION OF THE DRAWINGS

FIG. 1 shows a timeline for a method of capture of magnetic resonance imaging (MRI) data;

FIG. 2 illustrates a mismatch of contrasts between iNAV images; and

FIG. 3 illustrates sets of image data associated with an embodiment when applied to a process such as phase-sensitive inversion recovery (PSIR) that operates on two separate segmented acquisitions.

### DETAILED DESCRIPTION

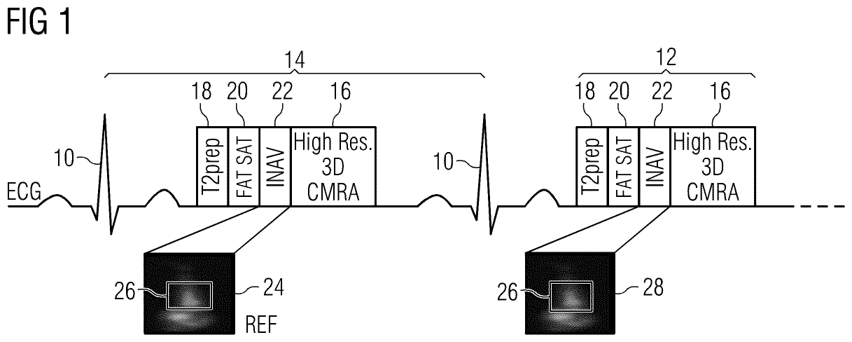

FIG. 1 shows a timeline for a method of capture of magnetic resonance imaging (MRI) data, as may benefit from application of the present embodiments. Such method of data capture may be referred to as “segmented acquisition”, in that shots containing one or more segments of MRI data are acquired during each cardiac cycle and combined together into a single image at the end of the acquisition.

A trace 10 illustrates an electrocardiogram measurement, indicating cardiac activity. As is conventional, image capture 12 is performed only in times that correspond to little cardiac activity. As illustrated, image capture acts 12 are provided within each cardiac cycle 14.

As schematically represented in FIG. 1, MRI raw data for a single image is captured in a number of shots 16, distributed in a number of cardiac cycles 14. By capturing the shot 16 of MRI raw data at a corresponding time within each cardiac cycle, effects of cardiac movement of the patient are largely removed. However, a cycle of respiratory motion by the patient extends over a number of cardiac cycles 14, and in order to combine MRI raw data from a number of cardiac cycles into a single MRI image, some compensation is to be applied for the respiratory motion.

A shot 16 of capturing MRI raw data is performed during a period of little cardiac activity. As only a short time is available, only a limited amount of data may be captured during each shot 16 and each cardiac cycle 14. While the timing of the shot 16 within the cardiac cycle 14 reduces any effects of the cardiac cycle on data capture, patient movement due to the respiratory cycle may cause disruption to image capture.

As is conventional, some preparatory steps may be taken in each cardiac cycle 14 prior to the shot 16 of MRI raw data capture. In the illustrated example of FIG. 1, first, a step of T2-preparation 18 may be performed in order to enhance blood signal, followed by a fat saturation pulse 20, which magnetically saturates fat signal within the patient, such that a signal from fatty tissue is minimized in the captured image data.

Next, in an act 22 temporally adjacent to the shot 16 of MRI data capture, data for a navigation image iNAV is captured in a navigation image act 22. The navigation image capture act 22 provides capture of a complete image, at relatively low resolution, representing a part of a patient in which respiratory motion may be observed. Since a cycle of respiratory motion will extend over many cycles of cardiac motion, the navigation image iNAV may provide an indication of the point in the respiratory cycle corresponding to the time at which MRI data was captured in the adjacent shot 16. An example of an iNAV navigation image is shown at 24. Within the iNAV image, a region of interest (ROI) is identified and highlighted with a box outline 26. The ROI is selected to represent a part of the body of the patient that is being imaged by the MRI data capture process, and that moves cyclically with the respiratory cycle. The ROI in the box window 26 is tracked over time by matching the iNAV signal from each data capture against a certain reference iNAV (e.g., the first one 24, labelled “REF”). Matching is performed by successively shifting the box 26 from the reference iNAV 24 REF around a pre-determined search window on the iNAV image to be processed 28. The content of the box 26 is compared to the reference REF for each shift using some intensity-based measure of similarity, such as normalized cross-correlation (NCC). The shift corresponding to the greatest measure of similarity is interpreted as a quantitative measure of the displacement of the ROI and is saved for each respective shot 16 of the MRI acquisition. Depending on whether a 2D or 3D iNAV technique is being used, the displacement information for each shot may be represented by a vector of either 2 or 3 values of translation. In one embodiment, the selection of the ROI and the orientation of the imaging contain the main direction of respiratory motion in one single dimension (e.g., Feet-Head (FH)).

The respiratory displacement of the ROI in each iNAV image is taken to indicate the displacement of the tissue subjected to MRI imaging data acquisition, and raw data obtained during each shot 16 may be compensated for translational motion by applying the corresponding translations as a linear phase shift to ultimately allow reconstruction of a motion-compensated image.

The captured iNAV images are of low resolution, to provide that capture of the iNAV images does not take too much time within each cardiac cycle. For that reason, it is difficult to perform the described matching using similarity measures that do not rely on image intensity but on structural information, such as mutual information. This provides that it is usually not possible to reliably perform iNAV-based motion compensation in the presence of varying contrast in the underlying MRI image acquisition, leading to varying contrast of the iNAV images to be matched.

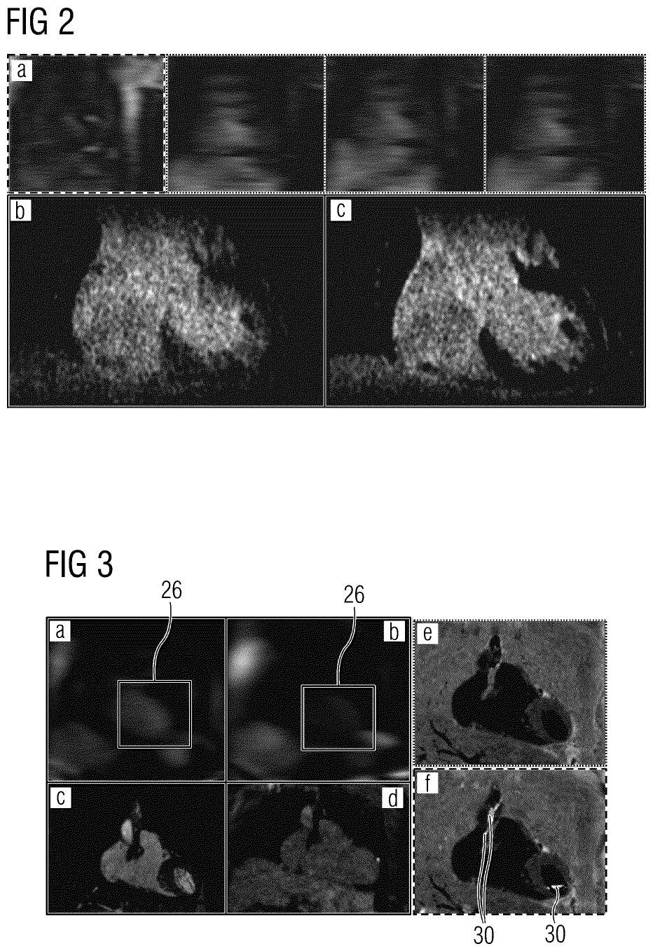

The situation of varying contrast may arise, for example, in case of ECG-triggered inversion-recovery scans of arhythmic patients. Missed, or incorrectly timed, triggers will lead to different inversion times with changed, or possibly inverted, image contrast (see FIG. 2a).

Also, possibly in addition to this first scenario, in some techniques, multiple segmented scans may be required, with different contrasts, to be aligned and to create a final image. For example, in phase-sensitive inversion recovery (PSIR) or relaxation parameter mapping. PSIR techniques rely on two different contrasts acquired at different inversion times with different excitation flip angles. Similarly, relaxation parameter mapping techniques sample the inversion curve along different inversion times, resulting in images with different contrasts. It will be necessary to align the captured MRI raw data from all shots 16 within each contrast (e.g., image), and further, it will be necessary to align the two contrasts (e.g., images) together. Within each contrast (e.g., image), respiratory motion is to be taken into account with respect to some reference position when assembling the MRI raw data from respective shots 16; and between contrasts (e.g., images), differences in the position of the respective reference positions are to be taken into account to allow the separate contrasts (e.g., images) to be combined/compared.

In the example case of an ECG-gated inversion-recovery acquisition with missed triggers, conventionally, data may be rejected after the matching act according to the motion values themselves. However, the assumption that a matching between unsuitable iNAV pairs will always lead to easily identifiable motion value outliers outside of the conceivable range is unreliable. Further, the reference iNAV may be chosen such that the reference iNAV followed a missed trigger with inverted contrast (see FIG. 2a). This leads to many, or potentially all, motion values to be derived from matching between iNAVs with different contrasts and will render the motion values meaningless.

Conventionally, in the case where multiple contrasts (e.g., images) are acquired, a separate registration act using non-intensity-based methods may be implemented to compensate a difference in respiratory positions between these contrasts (e.g., images). Such multi-contrast registration tasks are, however, known to be challenging and may take considerable computing time.

According to an aspect of the present embodiments, motion compensation of MRI raw data captured in separate shots 16 is improved by identifying and potentially excluding shots 16 or clusters of shots having iNAV images that are measurably different from the iNAV images of the majority of shots or other clusters of shots. In contrast to conventional methods, this is done before the act of matching iNAVs and obtaining motion values, such that a matching between unsuitable pairs of iNAVs exhibiting different contrasts is avoided.

In an example method of the present embodiments, shots 16 of MRI raw data are captured as illustrated in FIG. 1, until all MRI data for all required contrasts (e.g., images) is captured. All associated iNAV images are stored.

A clustering act is then performed on the iNAV images for each contrast (e.g., image). In other words, a characteristic of the images is defined, and a statistical analysis is performed on the iNAV images according to the defined characteristic. The statistical analysis is used to identify different clusters of shots 16 of MRI raw data having iNAV images that differ measurably with respect to the defined characteristic. The shots 16 of MRI data corresponding to small clusters may be classified as outliers and may be excluded from reconstruction of the image. In this way, shots 16 of data, the capture of which was poorly timed, may be excluded from the reconstruction, and an improved overall image may be obtained.

The defined characteristic of iNAV images may be overall contrast or intensity, or a measure of timing derived from a potential ECG trace of the acquisition of iNAVs and corresponding shots 16. Contrast or intensity may be defined quantitatively using a signal-based metric such as normalized cross correlation (NCC) or the time between successive ECG triggering events. Any suitable clustering algorithm may be used for clustering the so-derived data (e.g., k-means), as will be apparent to those skilled in the art.

Once outliers or clusters have been identified for each contrast (e.g., image), the corresponding MRI raw data shots 16 may be discarded or may be included in the reconstruction. This decision may take into account features such as cluster size or distance between clusters according to some metric.

Once the identified outliers or clusters have been excluded from consideration, for the remaining iNAV images, representing shots 16 of MRI raw data, one or more clusters may remain. For each cluster, a motion value may be calculated for each remaining iNAV within the cluster with respect to a suitable defined reference. Such reference may, for example, be an iNAV image corresponding to the center of its respective cluster. Where a single segmented scan results in a single non-outlier cluster, the eventual image reconstruction may be improved by normalizing of motion values between the shots 16 represented by the iNAV images of the cluster by adding or subtracting, as appropriate, a difference between corresponding motion values (e.g., the average, maximum, or minimum motion values of the cluster). By such operation, the smallest possible overall phase shifting of MRI raw data is achieved, and a resultant, motion-compensated MRI image reconstruction may be improved.

Where a single segmented scan results in two or more clusters of comparable size, the eventual image reconstruction may be improved by normalizing of motion values between the shots 16 represented by the iNAV images of the respective clusters by adding or subtracting, as appropriate, a difference between corresponding motion values (e.g., the average, maximum, or minimum motion values of the respective clusters). By such operation, the clusters of iNAV will be brought to the same respiratory position, and a resultant, motion-compensated MRI image reconstruction may be improved.

Where multiple contrasts (e.g., images) are under consideration, each derived from a separate segmented scan, a normalization of motion values for each cluster within each single image (e.g., contrast) as described above will automatically lead to an alignment of the multiple contrasts (e.g., images), as long as respective clusters are internally normalized with the same statistical reference (e.g., the average, maximum, or minimum motion value of respective clusters).

According to certain embodiments, an increased robustness of segmented MR imaging may be achieved, using navigation images iNAV to indicate a motion value of each shot 16 of MRI raw data. Incorrect motion values may arise with respect to, for example, arrythmia-induced contrast variations during the scan.

More specifically, clustering of iNAV images according to their contrast before motion tracking provides the use of a good tracking reference and enables a simple pathway for cluster detection and potential outlier rejection.

An example of this advantage as provided by the present embodiments will be discussed below with particular reference to FIG. 2, which illustrates a mismatch of contrasts between iNAV images, and a selected reference iNAV image used for tracking may lead to dramatic degradation of the resulting motion correction if an inappropriate reference iNAV image is selected. Embodiments provide for such outlier iNAV images, and the associated MRI raw data shots to be excluded from reconstruction, and may also be excluded from use as a reference image.

In other embodiments, improved alignment may be obtained between images acquired in separate segmented scans. Alignment of such separate images may be required in processes such as PSIR and relaxation parameter mapping, as discussed briefly above. In PSIR, an image with restored magnetization polarity is calculated from an inversion-prepared image and a reference image. Misregistration between these images may lead to significant artifacts in the final result.

As long as the number of segments for each segmented acquisition is high enough so that their, for example, average respiratory position may be assumed to be the same, the proposed approach is a simple and a more robust alternative to an additional implementation of non-intensity based image registration.

An example of the application of the present invention to PSIR will be discussed below with reference to FIG. 3.

FIG. 3 represents sets of image data associated with an embodiment when applied to a process such as PSIR that operates on two separate segmented acquisitions.

In FIG. 3, images a and b show navigation images iNAV for respective segmented acquisitions. Within each navigation image, a window 26 is shown, which visualizes the tracking of that part of the image representing the region of interest ROI. That part of the body of the patient corresponding to the ROI moves according to the respiratory cycle, and so the location of the window 26 may be interpreted as an indication of a current displacement of corresponding body parts due to the respiratory cycle.

Images c and d show the reconstructions of corresponding MRI image data of the inversion-prepared c and the reference image d. For both images, the reconstruction was internally motion-compensated using the corresponding iNAV series. Motion between the two images was unsuccessfully estimated based on matching the respective reference iNAVs of the two images, leading to the object of interest being shown in different respiratory positions in the two images.

Image f shows the result of a PSIR reconstruction based on the misaligned inversion-prepared image c and reference image d, showing numerous image artefacts 30, resulting from ineffective alignment of the two images.

Image e shows an improved PSIR reconstruction, resulting from improved alignment of inversion-prepared and reference image, using the described normalization based on an average of the cluster values of the two acquisitions.

In a feature of the present embodiments, iNAV images are analyzed, and outliers are identified. The shots 16 of MRI image data corresponding to the identified outliers are excluded from reconstruction of the MRI image. The remaining MRI image data will have similar iNAV motion estimation values, and average values for each image will be more representative of the remaining shots 16 of data. Therefore, the average iNAV motion estimation value will provide an improved localization of the respective images.

Turning to FIG. 2, part a shows a sequence of four iNAV images, each corresponding to a shot of MRI raw data. In a conventional arrangement, the first of these images may be taken as a reference, and a motion parameter of second, third, and fourth shots is calculated, with reference to the first shot, by performing an NCC-based matching between the respective second, third, or fourth iNAV navigation image, and the first iNAV navigation image, which is taken as a reference. However, as is clear to the human observer, the first iNAV image is very different from the second, third, and fourth iNAV images; the second, third, and fourth iNAV images are similar to one another. This may result from an arrythmia in the patient, causing the first iNAV image to be captured at an unsuitable magnetization state compared to the steady-state shown in the second, third, and fourth iNAV images.

Conventionally, the system will attempt to align each of the second, third, and fourth iNAV images to the first, and apply a corresponding motion correction to the respective MRI imaging data acquired in the adjacent shot 16. As the first iNAV image is clearly inappropriate for use as a reference image, these motion values and the corresponding corrections will be incorrect. When the shots 16 are assembled together, an image b may result. This image is not clear as a result of the failed motion correction.

According to the present embodiments, the iNAV images shown at a would be subjected to a clustering act. The first iNAV image would be identified as an outlier, or part of a cluster of outliers, being significantly different from the others. That iNAV image, and possibly also the associated shot 16 of MRI imaging data, would be excluded from further treatment. The second iNAV image may be selected as the reference image. Then, third and fourth iNAV images may be compared more effectively with the second iNAV image, as they are more similar to one another than the first iNAV image was. Improved motion value calculation will result, and the associated reconstructed image will resemble image c, showing much improved clarity as compared to the image b.

The images illustrated in FIG. 2 represent 3D Late Gadolinium Enhancement images of the myocardium using iNAV navigation images, showing the impact of a suboptimal choice of reference iNAV image for motion tracking. Image a shows iNAV images belonging to the first four shots of a 3D scan. In the illustrated example, the first iNAV image was preceded by a missed ECG trigger and was therefore captured at an inappropriate magnetization state with respect to the steady-state during the inversion-prepared exam; as a result, the first iNAV image exhibits an inverted contrast compared to the expected one exhibited by the second to fourth iNAV images.

Motion tracking with the first iNAV image as a reference led to failure of the motion correction, resulting in image b, whereas application of a method according to the present embodiments led to a better choice of tracking reference and an improved motion correction result as shown in image c.

The present embodiments may usefully be applied to situations in which segmented MRI acquisition is used in patients with significant arrythmia, or where ECG mis-triggering occurs for other reasons. The present embodiments provide iNAV image correction techniques than enable effective MRI reconstruction even despite such problems. The present embodiments may also or alternatively be used to provide improved registration and alignment between images captured in separate segmented scans, with iNAV image registration being applied between the images, despite exhibiting different contrasts or employing differing contrast preparations.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for reconstructing a set of one or more magnetic resonance imaging (MRI) images from one or more segmented acquisitions of MRI raw data, each of the one or more segmented acquisitions including a plurality of shots, the method comprising:
for each respective shot of the plurality of shots capturing a part of the MRI raw data of the respective MRI images, capturing and reconstructing a low-resolution navigation image of a region of interest, either from the part of the MRI raw data or from additional MRI raw data acquired adjacent to the part of the MRI raw data in time;
clustering navigation images from the plurality of shots within each MRI image of the one or more MRI images according to a characteristic of the navigation images;
excluding a navigation image of the navigation images and a corresponding part of the MRI raw data from further processing when the navigation image is identified as an outlier in the clustering;
estimating motion values of the object of interest from each remaining navigation image with respect to a reference navigation image within the respective cluster;
normalizing the motion values for all data clusters across the one or more MRI images within the acquired set of one or more MRI images; and
performing a final reconstruction of the set of one or more MRI images using the motion values.

2. The method of claim 1, wherein each shot of the plurality of shots is captured at a corresponding point in a cardiac cycle.

3. The method of claim 2, wherein the characteristic for clustering the navigation images is a time of capture within a cardiac cycle.

4. The method of claim 1, wherein the characteristic for clustering the navigation images is a characteristic of a navigation image signal.

5. The method of claim 4, wherein the characteristic of the navigation image signal for clustering the navigation images is a normalized cross-correlation.

6. The method of claim 1, wherein clusters of the navigation images are identified as outliers.

7. The method of claim 1, wherein estimating motion values of the object of interest is performed using a normalized cross-correlation of image signals of regions of interest within the navigation images.

8. The method of claim 1, wherein the reference navigation image for estimating motion values of the object of interest is identified as having a value of the characteristic corresponding to a center of values of the navigation images of the respective cluster.

9. The method of claim 1, wherein the normalizing of motion values between clusters within and across at least one MRI image of the one or more MRI images comprises, for each cluster in each MRI image of the at least one MRI image, subtracting a motion value representing an average motion position from all the motion values in the respective cluster.

10. The method of claim 1, wherein normalizing the motion values between clusters within and across at least on MRI image of the one or more MRI images comprises, for each cluster in each MRI image of the at least one MRI image, subtracting a motion value representing an end-expiratory motion position from all motion values in the respective cluster.

11. The method of claim 9, wherein the set of one or more MRI images includes a single MRI image for which a single non-outlier cluster is identified.
